(19) United States
(12) Patent Application Publication
(10) Pub. No.: US 2008/0173301 A1
(43) Pub. Date: Jul. 24, 2008

(54) VARIABLE DOSE AEROSOL DRUG CANISTER

(76) Inventors: Daniel Deaton, Apex, NC (US); Robert Williams, Raleigh, NC (US); Matthew D. Khare, Willow Spring, NC (US); Perry Genova, Chapel Hill, NC (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(21) Appl. No.: 11/850,177

(22) Filed: Sep. 5, 2007

Related U.S. Application Data
(60) Provisional application No. 60/842,552, filed on Sep. 6, 2006.

(51) Int. Cl. A61M 16/20

(52) U.S. Cl. 128/203.12

(57) ABSTRACT

A variable volume medicament valve, inhaler; and method of treatment including a housing, and a plug insertable into the housing wherein a volume of medicament to be released by the valve is defined by a distance between a lower surface of the plug and a top surface of the housing and wherein the volume is variable.

### CROSS-REFERENCE SECTION TO RELATED APPLICATIONS

This application claims the benefit of U.S. Provisional Patent Application No. 60/842,552, filed Sep. 6, 2006, which is hereby incorporated by reference in its entirety.

### FIELD OF THE INVENTION

The present invention relates to metered dose aerosol canister and in particular to a metered dose aerosol canister providing a variable valve for administration of desired amounts of drug depending upon need.

### BACKGROUND OF THE INVENTION

Diabetes is a disease affecting million of people in the United States and millions more world-wide. For diagnosed diabetics a common treatment is the use of insulin therapy. Generally, a diabetic patient self-administers insulin by injection. Diabetes is one of the few diseases where doctors routinely allow the patient to self titrate, that is, determine the exact amount of dosage to deliver to themselves at any give time. This variability of dose creates problems when attempting to deliver the dose through means other than the traditional injection.

In an effort to provide for a non-invasive means of administering insulin and other systemic drugs, and thereby eliminate the need for syringes, aerosolized formulations have been theorized.

Heretofore, the studies and experiments in the pulmonary delivery of insulin have suffered from poor reproducibility of the dose to be inhaled. Typically, the known inhaler devices use a non-metered canister which, when the valve is depressed, continues to deliver its dose until the valve is released. In order to achieve variable dosing, it is necessary for the device to have complex circuitry and relays for electrical control of the valve. While functional, these devices are not commercially viable due to the expensive and heavy batteries. In addition, the packaging of all of these items leads to a rather large and unwieldy inhaler.

Aside from diabetes and its treatment with insulin, a number of other diseases require the active participation and understanding of the patient to provide for effective dosing. For example, many people suffer from chronic obstructive pulmonary disease (COPD) and asthma.

Thus there is a need for a device and method providing for the effective and variable dosing for a patient to insure that effective amounts of drug are received at the desired time. There is a further need that such a device is user friendly providing adequate administration of the drug preferably in a single inhalation.

The present invention endeavors to overcome the problems of the prior art and provide a non-invasive device and methodology for delivery of drugs that produces repeatable and variable/controlled dosage amounts of a drug to the patient substantially without the need for complex circuitry having high energy demands.

### SUMMARY OF THE INVENTION

One aspect of the instant invention is directed to a variable volume medicament valve including a housing; and a plug insertable into the housing wherein a volume of medicament to be released by the valve is defined by a distance between a lower surface of the plug and a top surface of the housing and wherein the volume is variable.

Another aspect of the present invention is a variable dose metered inhaler including an inhaler body, a canister for storage of one or more pressurized doses of a medicament which is placed in the inhaler body, and a dose variable valve, the dose variable valve including a valve housing and a valve plug, wherein a volume of drug to be administered can be varied by movement of the valve plug in relation to the valve housing.

A further aspect of the instant invention is a breath actuated variable dose metered inhaler including an inhaler body having a mouthpiece, a canister for storage of one or more pressurized doses of a medicament which is placed in the inhaler body, and a dose variable valve. The dose variable valve includes a valve housing and a valve plug, wherein a volume of drug to be administered can be varied by movement of the valve plug in relation to the valve housing. The breath actuated variable dose metered inhaler also includes a breath actuated trigger which releases the volume of drug to be administered to the mouthpiece upon inhalation by a user.

Yet a further aspect of the instant invention is a variable dose metered nasal drug delivery device including a nasal drug delivery device body having a mouthpiece and a nosepiece, a canister for storage of one or more pressurized doses of a medicament, and a dose variable valve. The dose variable valve includes a valve housing and a valve plug, wherein a volume of drug to be administered can be varied by movement of the valve plug in relation to the valve housing. The variable dose metered nasal drug delivery device also includes a breath actuated trigger which releases the volume of drug to be administered to the nosepiece upon the exhalation of a user into the mouthpiece.

An additional aspect of the instant invention is a method of treatment by application of a drug effective for the treatment including steps of providing a breath actuated inhaler, providing a pressurized container having a medication, and setting a dose based upon the need of the patient. The method also includes steps of inhaling on a mouthpiece of the breath actuated inhaler, and delivering the set dose to the patient.

Another aspect of the instant invention is a method of treatment of a condition in need thereof by application of a drug effective for the treatment thereof including steps of providing a breath actuated nasal drug deliver device, providing a pressurized container having a medication, and setting a dose dependent upon the instant need of the patient. The method also includes steps of exhaling in a mouthpiece of the breath actuated nose drug delivery device, and delivering the set dose to the patient via a nose piece.

A further aspect of the present invention is a method of varying the volume of a valve for release of a pressurized material including steps of providing a variable volume medicament valve having a housing, and a plug insertable into the housing wherein a volume of medicament to be released by the valve is defined by a distance between a lower surface of the plug and a top surface of the housing, and rotating the housing relative to the plug to change the distance.

Further characteristics, features, and advantages of the present invention will be apparent upon consideration of the following detailed description of the invention taken in conjunction with the following drawings, and in which:

### BRIEF DESCRIPTION OF THE FIGURES

FIG. 1 is an exploded-view diagram of a variable valve according to one aspect of the present invention;

FIG. 2 is an assembled view of the variable valve as shown in FIG. 1;

FIG. 3 is a cross-sectional view of a metered-dose inhaler having a variable valve as shown in FIG. 1;

FIG. 4 is a cross-sectional view of a metered-dose inhaler having a variable valve as shown in FIG. 1 during discharge;

FIG. 5 is a cross-sectional view of a metered-dose inhaler having a variable valve as shown in FIG. 4, wherein the metered volume has been decreased;

FIG. 6 is a perspective view of an inhaler according to one aspect of the present invention;

FIG. 7 is a perspective view of an inhaler in the cocked position;

FIG. 8 is a perspective view of an inhaler showing two piece construction of the insertion of a drug canister;

FIG. 9 is a cross-sectional view of an inhaler according to one aspect of the present invention in the stored position;

FIG. 10 is a cross-sectional view of an inhaler according to one aspect of the present invention in the cocked position;

FIG. 11 is a cross-sectional view of an inhaler according to one aspect of the present invention at the point when the user begins to inhale;

FIG. 12 is a cross-sectional view of an inhaler according to one aspect of the present invention during administration of the drug;

FIG. 13 is a cross-sectional view of an inhaler according to one aspect of the present invention following administration and closure of the coves to return the device to its stored position;

FIG. 14 is a schematic diagram of an electrical circuit according to one aspect of the instant invention;

FIG. 15 is a cross-sectional view of an inhaler according to another aspect of the instant invention;

FIG. 16 is a cross-sectional view of a breath actuated nasal inhaler according to another aspect of the instant invention.

### DETAILED DESCRIPTION

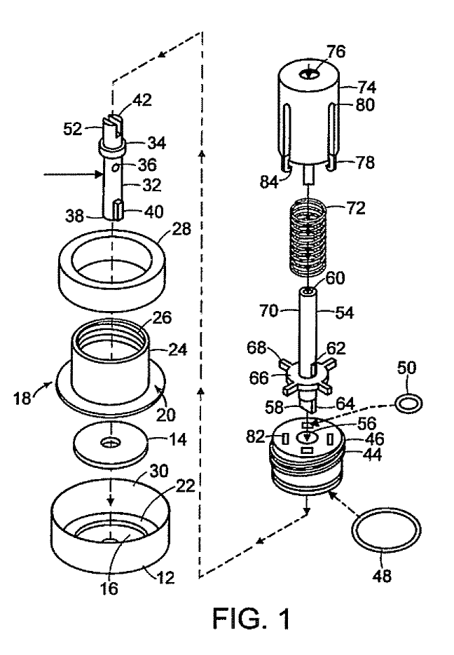

FIG. 1 shows a dose variable valve 10 according to a first aspect of the present invention. The dose variable valve 10 of FIG. 1 is shown in exploded form detailing its components. The dose variable valve 10 includes a ferrule 12, which enables the valve 10 to be attached to a canister (not shown in FIG. 1) containing drug medicament and propellant. Resting on an inner surface 16 of the ferrule 12 is a lower stem seal 14. The diameter of the inner surface 16 substantially conforms to the diameter of the lower stem seal 14. The lower stem seal 14 prevents leakage of medicament from escaping along the exterior of the shaft of the lower stem 32, when the valve 10 is actuated. The lower stem seal 14 also prevents debris and dirt from entering the valve 10. Resting on top of the lower stem seal 14 is a metering chamber housing 18. The metering chamber housing includes a lower flange 20, which when inserted into the ferrule 12 substantially conforms to the surface 22, and sandwiches the lower seal 14 between itself and the ferrule 12. The metering chamber housing also has a body 24, extending vertically away from the flange 20. The body 24 includes threads 26 formed on the interior surface of the body 24.

A gathering ring 28 rests on top of the flange 20 of the metering chamber housing 18 and between the body 24 of the metering dosing chamber and the side wall 30 of the ferrule 12. When installed on a canister (not shown) by crimping, the gathering ring 28 is compressed and forms an air tight seal between the ferrule and the canister to prevent escape of the pressurized medicament and propellant.

The lower stem 32 of the dose variable valve 10 is inserted into lower stem seal 14 up to the hub 34. The lower stem 32 extends through the ferrule 12 and also into the metering chamber housing 18. The lower stem 32 includes an orifice 36 that, as will be described below, allows medicament to flow from the metering chamber through the lower stem and be dispensed to the patient. The lower stem 32 is hollow and has an opening on the distal end 38, proximal to the tab 40. A lumen connects the opening at the distal end 38 to the orifice 36. The tab 40, as will be discussed below provides a point that can be grasped or fit in a keyway allowing the application of pressure to rotate the valve stem 32 to change the volume of the metering chamber. The lower stem 32 also includes a slot 42 for engaging the upper stem.

A variable metering plug 44 can be inserted into the meter chamber housing 18. The variable metering plug 44 includes threads that correspond to the threads 26 formed on the inside of the metering chamber housing. A metering chamber seal 48 can be affixed to a lower portion of the variable metering plug 44 and prevents the drug or propellant from entering the metering chamber between the metering chamber plug 44 and the metering chamber housing 18. This in reality is only a problem during discharge of the metering chamber, when the pressurized dose is suddenly exposed to the atmosphere via the lower stem 32. Without the seal 48, a larger dose than intended could escape from the canister and be administered to the patient. Obviously this would defeat the purpose of a dose variable valve. Having similar function is an upper stem seal The upper stem seal 50 sits in the variable metering plug 44 and forms a seal between the upper portion 52 of the lower stem seal 32 and the variable metering plug.

The upper stem 54 rests in an orifice 56 in the variable metering plug 44. The upper stem includes a lower portion 58 formed to interconnect with the slot 42 formed in the lower stem 32. This interconnection provides a continuous stem having a substantially constant diameter. The upper stem has a lumen 60 extending there through, which as will be explained below provides fluid communication between the canister and the metering chamber. The upper stem further includes an upper orifice and lower orifices 62 and 64, respectively. These orifices 62 and 64 also connect the canister to the metering chamber. Further aspects of the upper stem include a disk 66 located between the upper and lower orifices 62,64. Protruding from this disk are arms 68. As shown in FIG. 1, the disk 66 includes four arms, however, other arrangements of more or fewer arms are considered within the scope of the instant invention. Finally the upper stem includes shaft 70 extending from the disk 66. As stated above, the shaft 70 has a lumen 60 extending there through ending at the lower orifice 64.

A spring 72 is placed over the shaft 70 and rests on the disk 66. This spring provides the restorative bias that after actuation of the valve restores the valve to the at-rest position, as will be discussed in detail below.

A valve body 74 having an orifice 76, feet 78, and slots 80 is the final component of the dose variable valve. The valve body 74 is placed over the spring 72 that has been placed over the shaft 70. The valve body is lowered onto the variable metering plug 44, and the feet 78 engage holes 82 formed in the top surface of the variable metering plug 44. As shown in FIG. 1 the feet 78 have a projection 84 that secures them in the holes 82. Again, other means of securing the valve body to the variable metering plug 44 are considered within the scope the instant invention.

When the valve body 74 is placed in position on the variable metering plug 44, the shaft 70 of the upper stem 54 projects out the orifice 76. The interaction of the orifice 76 and the shaft 70 provides a vertical alignment mechanism for the valve 10. Also when placed in this position the spring 72 is slightly pre-tensioned, forcing the disk 66 firmly against the top surface of the variable metering plug 44. The arms 68 which extend from the disk 66 extend through the slots 80.

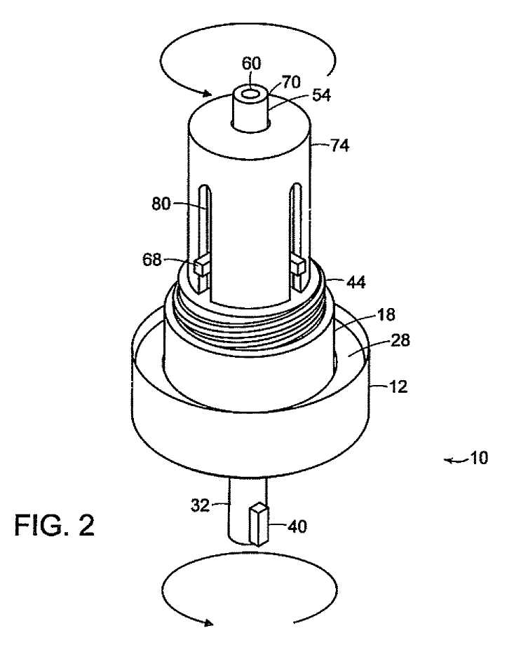

When assembled, the forgoing components take the shape of the dose variable valve as shown in FIG. 2. As shown in FIG. 2 the entire valve rests on the ferrule 12, with the lower stem 32 with tab 40 extending through the bottom of the ferrule 12. The gathering ring 28 is shown resting inside the ferrule 12 and holding the metering chamber housing 18 in the bottom of the ferrule 12. In practice, the gathering ring 28 itself does not apply this pressure keeping the metering chamber housing 18 in the bottom of the ferrule 12, but rather the pressure applied to the gathering ring 28 by the canister, when the valve 10 with the ferrule 12 is crimped thereon.

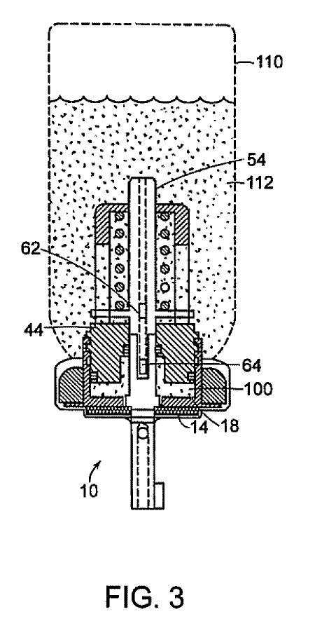

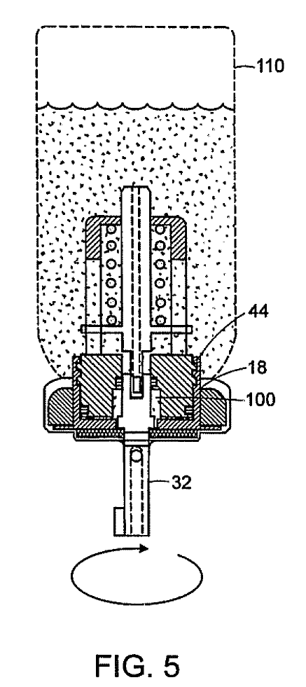

As assembled, the lower stem 32 translates through the ferrule 12 and into the metering chamber housing 18 to engage the lower portion 58 of the upper stem 54. The metering chamber housing 18 and the variable metering plug 44 are threaded together. The metering chamber 100 is defined by the orientation of these two components, the further into the metering chamber housing 18 the variable metering plug 44 is threaded the smaller the metering chamber 100. Comparing FIGS. 3 and 5 illustrates differences in metering chamber 100 dimensions.

Because in practice the metering chamber housing 18 is fixed firmly against the ferrule 12 it does not move when the lower stem 32 is rotated. Rotation of the lower stem 32 acts on the upper stem 54 because the two are joined by the lower portion 58 of the upper stern 54 and the slot 42 of the lower stem. The rotational force applied to the lower stem 32 is translated through the upper stem 54 to the valve body 74 by the arms 68 that engage the slots 80 formed in the sides of the valve body 74. This rotational force applied to the valve body 74 is similarly translated to the variable metering plug 44 via the feet 78 which engage the holes 82. As a result, rotation of the lower stem 32 causes a similar rotation of the variable metering plug 44. The variable metering plug 44 having threads 46 which mate with the threads 26 of the metering chamber housing 18 can rotate along these threads. Thus, by rotating the lower stern 32, the size of the metering chamber 100 can be changed.

FIG. 3 shows a cross-sectional view of a dose variable valve 10 inserted into a canister 110. The canister contains a drug and propellant suspension. As can be seen in this position of the variable valve a metering chamber 100 is formed between the bottom surface of the variable metering plug 44 and a top surface of the metering chamber housing 18. In this position, the canister 110 and the metering chamber 100 are in fluid communication via the upper and lower orifices 62 and 64 on the upper stem 54. Medicament 112 is prevented from escaping from the metering chamber 100 by the lower stem seal 14.

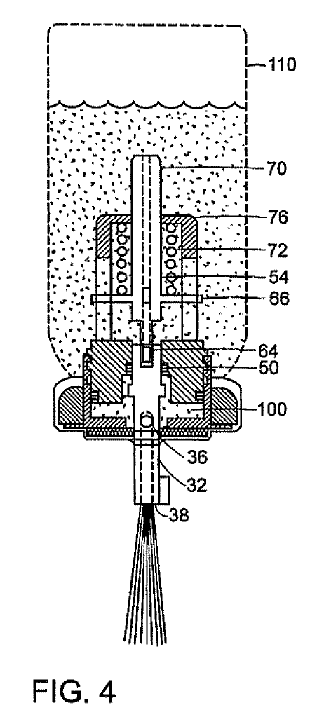

FIG. 4 depicts a cross-sectional view of a dose variable valve 10 inserted into a canister 110. In FIG. 4, the lower stem 32 has been depressed, forcing the stem into the valve 10 and exposing the orifice 36 to the metering chamber 100. The lumen connecting the orifice 36 to the distal end 38 of the lower stem 32 allows the pressurized suspension of drug and propellant to escape from the metering chamber 100 and be dispensed to the patient. Rapid expansion of the propellant which may for example be HFA 134a, caused by the pressure drop from inside the metering chamber to the ambient pressure provides the dispersant effect and causes the drug to be administered at the desired size for proper distribution to the lungs of the patient.

In the position shown in FIG. 4, the upper stem 54 is forced vertically upwards causing the lower orifice 64 to move vertically beyond the upper stem seal 50, thereby isolating the canister 110 from the metering chamber 100. The spring 72 is compressed by the movement of the upper stem 54 which compresses the spring 72 between the disk 66 and the valve body 76. The spring 72 provides the restorative force to return the valve 10 to its resting position once the force applied to the lower stem 32 is released. Once released, the valve 10 returns to the position shown in FIG. 3, the metering chamber 100 is once again in fluid communication with the canister 110 and a dose of medicament flows form the canister 110 to the metering chamber 100 where it sits until a further dose is administered.

Rotating the lower stem 32 as shown in FIG. 5 effects a change in the dose to be administered to the patient. A comparison of FIGS. 3 and 5 shows that when holding the canister 110 in the position shown, a counter-clockwise rotation of the lower stem 32 causes the variable metering plug 44 to be threaded into the metering chamber housing 18 causing the metering chamber 100 to be made smaller.

The dose variable valve 10, as shown above, allows the patient to set the precise amount of drug prescribed or deemed necessary at any particular time. The dose can be administered in a single inhalation, such that in certain instances multiple inhalations are generally not necessary. For example, the dose variable valve 10 may provide variable dosages from about 25 to 300 μl. In one preferred embodiment, the dosage from 25 to 300 μl can be accomplished in 12 equal 25 μl steps allowing for very precise and repeatable titration of dosage for the patient.

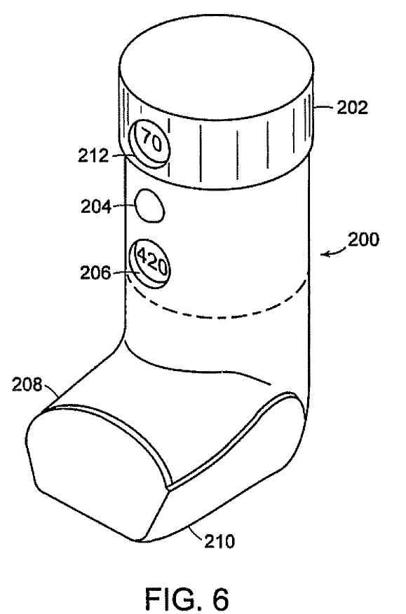

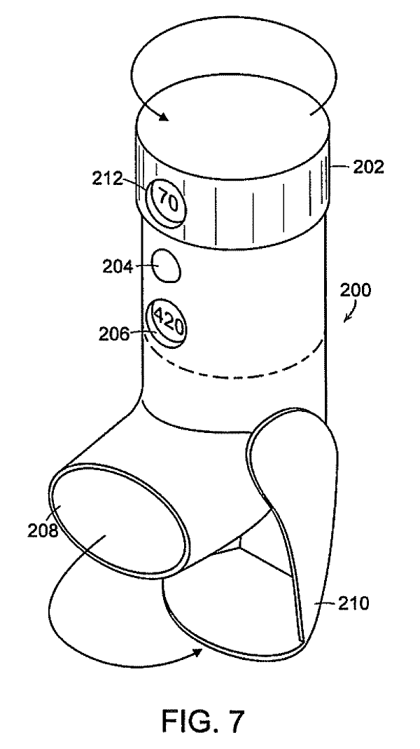

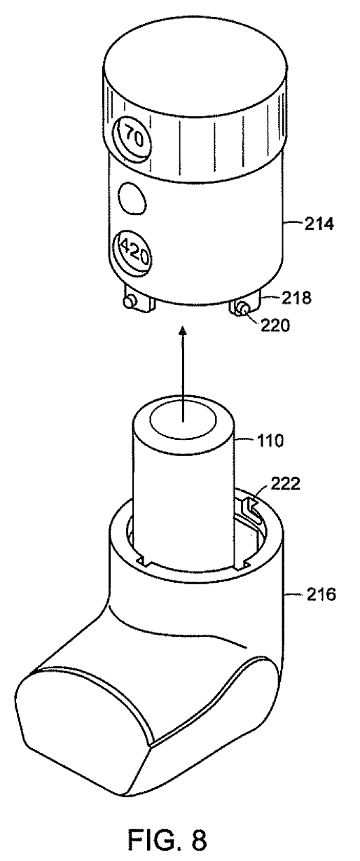

A variable dose inhaler 200 incorporating the dose variable valve 10, according to another aspect of the instant invention is depicted in FIG. 6. The inhaler 200 includes a dose variable valve (not shown) and canister 110 combination as described above, and shown in FIG. 8. On the exterior of the inhaler 200 is a dial 202 which may be turned by the user to alter the dose to be administered by the inhaler 200. The inhaler also includes a LED light 204 that indicates to the user when the medication is being dispensed. The inhaler 200 is a breath-actuated inhaler where the inhalation of the patient triggers the release of the medication. In such an application, the LED light 204 turns on upon cocking of the device as shown in the progression from FIG. 6 to FIG. 7, and continues to be illuminated as the device administers a dose, turning off once the dose is administered to signal the user they can cease inhaling. Details of this are described further below.

The inhaler 200 includes a units remaining indicator 206. As can be readily understood by those of skill in the art, in a device that has variable dosing characteristics, the accounting for the volume of medicament already administered and the amount of medication remaining in the device are important so that the patient is never in a situation where there are no doses remaining when they are in need of the medication. In practice, doses of medication are not counted as in the traditional dose counters but rather IU's or inhalation units are counted. As a result when a patient determines that they require a dose of 70 inhalation units as shown in FIGS. 6 and 7, the user twists the dial 202 until the dose setting indicator 212 reads 70. The user then rotates the cover 210 to expose the mouthpiece 208. At this position, the inhaler 200 is in position to administer a dose of 70 IU's to the patient. Upon administration, of the 70 IU's, the value will be subtracted from the number of IU's indicated as remaining in the inhaler by IU's remaining display 206.

FIG. 8 shows the inhaler 200 opened into its two component parts, a base 216 and a cap 214. The cap 214 can be joined to the base 216 by any suitable means known to those of skill in the art. As shown in FIG. 8, tabs 218 are formed on the cap 214 and include projections 220 extending outwardly therefrom. The projections fit into slots 222 formed in the base 216. The slots may, as shown, have and L-shape allowing the cap 214 to be rotated into the slot 222 and secured to the base 216.

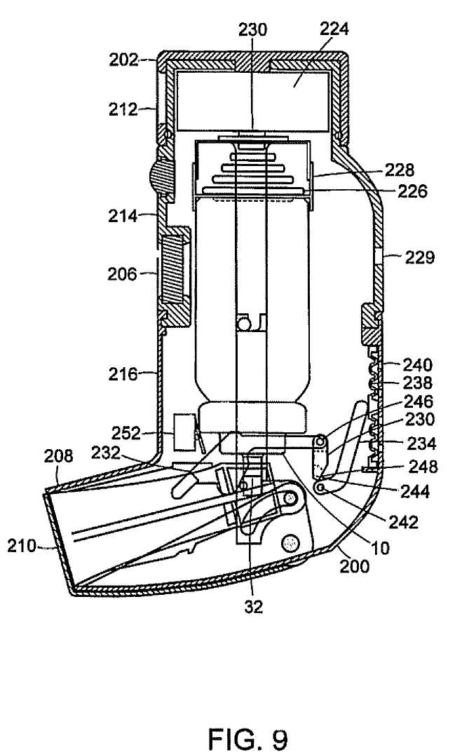

The following description is of a breath-actuated inhaler incorporating the dose variable valve and canister described above. FIG. 9 shows a cross sectional view of a breath actuated inhaler 200 according to one aspect of the instant invention. The inhaler 200 includes a canister 110 and a dose variable valve 10. The canister 110 and dose variable valve 10 are joined together to form a single unit as shown in FIGS. 3-5. As shown in FIG. 9 the inhaler is in the stored or at-rest position. The mouthpiece 208 is covered by a hinged cover 210, which, as will be discussed, also acts as the cocking mechanism for the inhaler 200. The inhaler 200 is formed of at least two pieces, a base 216 and a cap 214. The cap 214 includes an IU's remaining display 206, which is preferably an LCD, liquid crystal display. The cap 214 also includes a inhalation light 204, which may be an LED, light emitting diode, and a dose setting indicator 212 which indicates the number of IU's to be administered as a dose, as with the IU's remaining this may also be an LCD, but may also be a simple printed indicator, which is uncovered as the dial 202 is rotated.

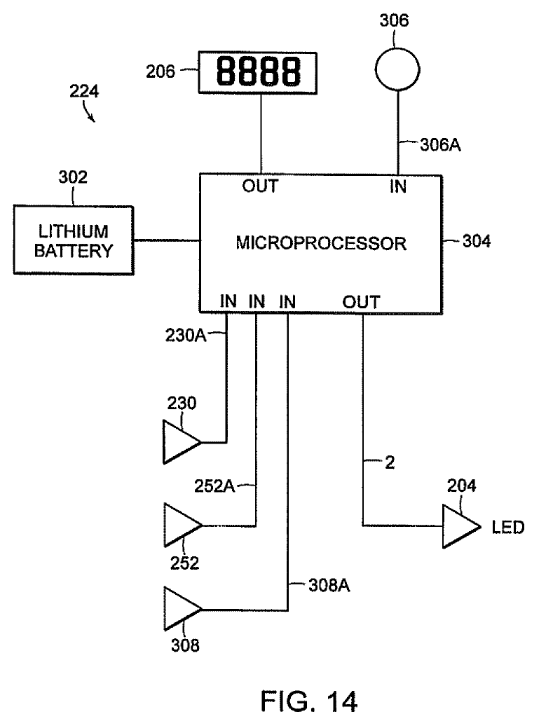

The inhaler 200 shown in FIG. 9 also includes a computer 224 comprised of two portions. The first portion is a battery which provides power to the inhaler for the LED and LCD's and the second portion is a microprocessor which is shown in more detail in FIG. 14 and will be discussed below.

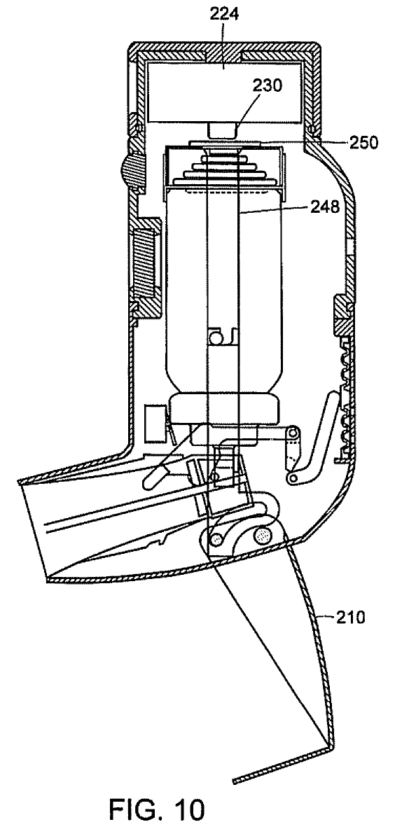

The cap 214 contains a spring 226 that rests in a spring holder 228 the spring holder 228 is keyed or affixed to the canister 110. The cap 214 also contains a cocking switch 230 which provides an electrical signal to the microprocessor when it senses that the inhaler has moved from the at-rest position shown in FIG. 9 to the cocked position shown in FIG. 10. A further feature of the cap 214 is an orifice 229 that allows for the entry of air from the atmosphere into the inhaler 200 that assists in dispersing the medicament upon administration and providing a volume to be inhaled with the medicament by the user.

The cap 214 includes a dial 202. The dial 202 is preferably mechanically connected to the canister 110. By this mechanical connection, rotation of the dial 202 rotates the canister 110. The lower stem 32 of the dose variable valve 10 is keyed to the base 216 of the inhaler 200 via, for example, the inhaler nozzle, to prevent its movement. As a result of the lower stem 32 being prevented from moving, rotation of the dial 202 forces the canister 110 to rotate in relation to the lower stem 32. As discussed above, rotation of the lower stem 32 in relation to the canister 110 changes the relationship between the variable metering plug 44 and the metering chamber housing 18 in the dose variable valve 10. As a result the user is able to adjust the size of a dose of medicament the inhaler 200 is to administer.

The inhaler includes a release mechanism which includes a rocker 232, a cam 234, a follower, 236, and a diaphragm 238. In FIG. 9, the cam 234 is connected on one end to the diaphragm 238, and is held in place by a pin 242 on the other end. The cam 234 also includes a lip 244 which is formed on the end of the cam 234 connected to the pin 242. The follower 236 is connected to the rocker 232 by another pin 246; the follower 236 is free to rotate about the pin 246. In addition, the follower 236 has a lip 248 which interacts with the lip 244 as will be discussed below.

As mentioned above, FIG. 9 shows the inhaler 200 in the at-rest position. In this position the spring 226 is in a less biased state and the spring holder 228 is expanded. The cocking switch 230 is depressed and the follower 236 rests on the cam 234.

As shown in FIG. 10, the cover 210 has been opened exposing the mouthpiece 208. The movement of the cover 210 has caused a rod 248 having a head 250 located on the top surface of the spring holder 228 to move in the direction of the base 216. The movement of the rod 248 compresses the spring holder 228 and the spring 226 placing the spring in a more biased position. The movement of the spring holder 228 causes the cocking switch 230 to move. The movement of the cocking switch sends a signal to the microprocessor indicating that the inhaler 200 is now in a cocked position.

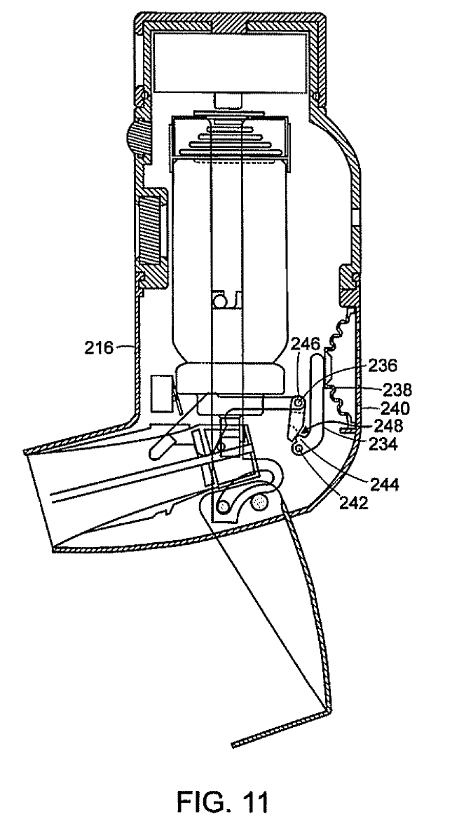

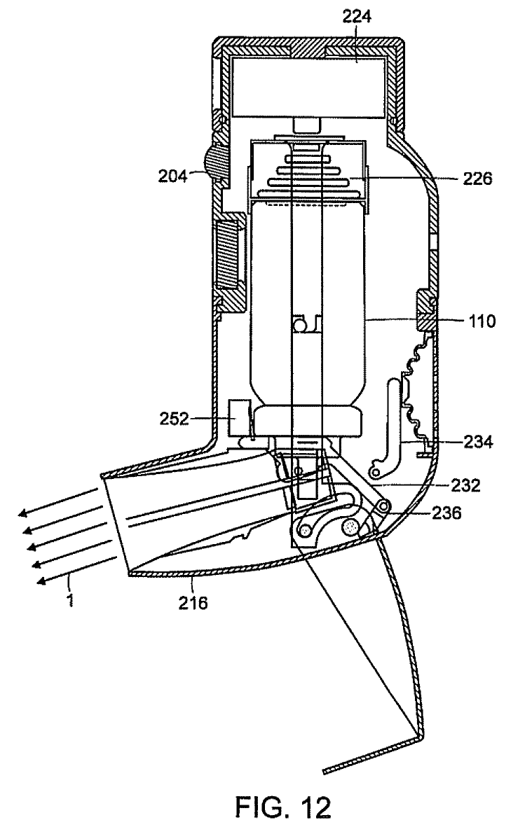

In FIG. 11, the patient begins to inhale. The patient's inhalation causes a vacuum to develop internally in the inhaler. This vacuum causes the diaphragm 238 to deform in the direction of the user's mouth, which is the origin of the vacuum. The deformation of the diaphragm is assisted by holes 240 formed in the base 216 which allow air to enter the base on a backside of the diaphragm. This air that enters the backside of the diaphragm 238 is of a higher pressure than the vacuum created internally in the inhaler 200, which thereby causes the deformation of the diaphragm 238. The movement of the diaphragm 238 causes movement of the cam 234 about the pin 242. The movement of the cam 234 causes the lip 244 formed on the cam 234 to put pressure on the lip 248 of the follower 236. The follower 236 begins to rotate about the pin 246 connected to the rocker 232 because the rocker is held in place by the canister 110. As the diaphragm 238 continues to expand, lip 244 formed on the cam 234 forces the follower 236 off of the cam 234 as shown in FIG. 12.

Upon release of the follower 236 from the cam 234, the rocker 232 is free to pivot. With respect to FIG. 12 the rotation is in a clock-wise direction. The movement of the rocker 232 releases the canister 110 and allows the spring 226 to expand by forcing the canister 110 to move in the direction of the base 216. As described above, the stem 32 of the valve 10 is in slidable engagement with the canister 110. As a result the canister 110 slides over a portion of the stem 32 of the valve 10 allowing the canister 110 to move in the direction of the base 216. This movement, as described above, allows an orifice in the stem 32 of the valve 10 to be exposed to the pressurized dose in the metering chamber 100 and thereby release the pressurized dose 1 through a lumen formed in the stem and to exit the mouthpiece 208.

The expansion of the spring 226 is enabled by the release of the rocker 232. The spring 226 acts on the canister 110 on one side and against the spring holder 228 on the other side. The top portion of the spring holder 228 is held in pace by the rod 248 which is connected to the cover 210. The head 250 of the rod 248 prevents the expansion of the spring holder, 228 in the direction of the cap 214. The spring holder 228 does expand in the direction of the canister 110 as shown in FIGS. 11-12. The spring 226, as shown in FIG. 12, is in the less biased position shown originally in FIG. 9.

The movement of the canister 110 also causes an actuator sensor switch 252 to close, as can be seen by comparison of FIGS. 11-12. The closure of the actuator sensor switch 252 sends a signal to the computer 224 to running of a clock function within the computer 224. The clock function as will be described below continues sending power to illuminate the LED 204 for a time specified depending on for example the dose to be administered. Upon the running of the clock for the time specified the LED will no longer illuminate signaling to the user that they can stop inhaling as the dose has been administered by the inhaler 200. Other functions of the actuator sensor switch 252 and the computer 224 are discussed below with respect to FIG. 14.

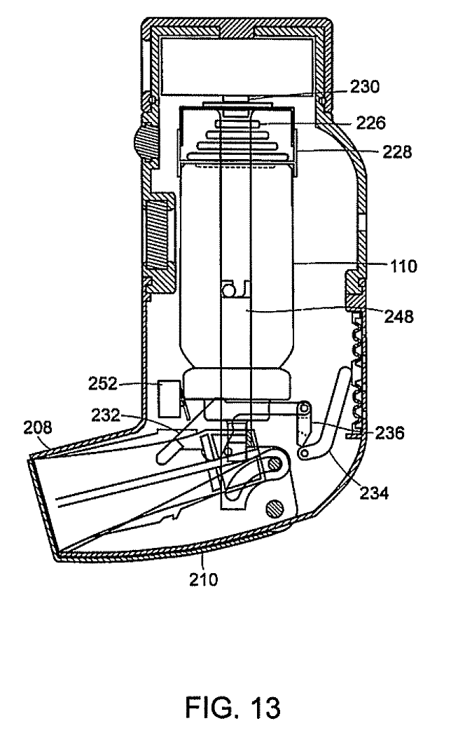

FIG. 13 shows the return of the inhaler 200 to the at-rest position. The cover 210 is closed covering the mouthpiece 208. The movement of the cover 210 acts on the rod 248 to move the canister 110 in the direction of the cap 214. The movement of the canister 110 acts on the spring holder 228 which is keyed, or affixed, to the canister 110. The top portion of the spring holder 228 contacts the cocking switch 230 and depresses it sending a signal to the computer 224. The movement of the canister 110 allows the spring 72 of the dose variable valve 10 shown in FIG. 1, to extend the lower stem 32 of the dose variable valve 10 and return it to an at-rest position. The rocker 232 is returned to its at-rest position: which in turn returns the follower 236 onto the cam 234, which has also returned to its at-rest position once the pressure inside the inhaler and the pressure out side the inhaler have equalized following administration of the dose. This may be assisted by making the diaphragm 238 of a material that is biased in the direction away from cam 234. The movement of the canister 110 also removes the pressure applied to the actuator sensor switch 252.

The computer 224 will now be discussed with respect to FIG. 14. The computer includes a battery 302 and a microprocessor 304. The battery 302 supplies power to the electrical components of the inhaler 200 including the LED 204, the LCD 206, and the switches, as well as the microprocessor 304. The microprocessor 304 receives inputs from several sources including the dose selector 306, which is formed by the dial 202 and a number of electrical contacts on the cap 214. By rotating the dial 202, a different electrical contact (not shown) on the cap 214 is contacted by an electrical contact (not shown) in the dial 202 causing a circuit to be formed. The parameters of this circuit provide an input 306A to be supplied to the microprocessor 304 from the dose selector 306. The dose selector signal 306A supplied to the microprocessor 304 is used to determine the time period for a clock signal. As will be discussed below, the clock signal is used to provide a time period during which the LED 204 will be illuminated following triggering of the inhaler 200 to release a dose. This does signal 306A also indicates the number of IU's to be deducted from the IU's remaining LCD 206.

Another input received by the microprocessor is the cocking switch input 230A which is received once the inhaler has been cocked as shown in FIG. 10. In one embodiment this may be a normally closed switch, such that when the pressure applied by the canister 110 is removed, returns to its closed position to complete the circuit. This may be for example, to act as an on/off switch for the inhaler 200 so that power is conserved at all times except when the device is cocked and ready to administer the drug. As a result, when the inhaler 200 is returned to the at-rest position as shown in FIG. 13, following administration of the drug, the power to the inhaler is shut off except for the display of the IU's remaining 206 which may be constantly maintained.

The actuation sensor switch 252 or inhalation switch sends a signal 252A when it is depressed. This typically coincides with a user inhaling on the mouthpiece 208. This inhalation, as discussed above, triggers the inhaler to dispense the drug. Triggering causes the canister 210 to move in the direction of the base 216 and close the normally open actuation sensor switch 252. The closure of this switch sends a signal to the microprocessor 304 to start a clock signal that controls the illumination of the LED 204. The LED will be illuminated for as long as the microprocessor 304 has determined necessary for the desired dose. As described above, the microprocessor 304 performs a calculation based on the dose selector signal 306A. Once the actuation sensor switch 252 is switched on, the LED will be illuminated signaling to the user to continue inhalation. Upon expiry or the running of the clock to zero, the microprocessor 304 opens the circuit to the LED 204 extinguishing the light and signaling the user that they can stop inhaling.

Another feature of the actuation sensor switch 252 is that upon depressing the switch, the microprocessor 304 is signaled that a dose is being expelled from the canister 110. The amount of that dose is signaled by the dose selector 306. The microprocessor 304 uses the dose signal 306A in combination with the closing of the actuation sensor switch 252 to perform a calculation and deduct the amount of the dose from the IU's remaining LCD display 206. This may be done upon triggering of the sensor switch 252, or at a later time, for example, when the inhaler 200 is returned to its at-rest position shown in FIG. 14.

The microprocessor 304 also receives an input from a temperature sensor 308 which provides a temperature signal 308A. As will be appreciated by those of skill in the art, the dispensation of a pressurized medicament as used in an inhaler will be affected by the temperature of the inhaler, which is generally near the ambient temperature. The higher the temperature, the higher the pressure that will be developed by the expansion of the propellant inside the canister 110.

By use of the forgoing microprocessor 304 as described with respect to FIG. 14, the user of the inhaler 200 is able to dial in the exact dose to be administered. In the preferred embodiment these will be in approximately 25 μl increments and include the range of 25-300 μl. In such a configuration the dial 202 will have 12 stops one each for doses from 25-300 μl, one approximately every 30 degrees around the perimeter of the dial 202.

The microprocessor enables the user to set their desired dose and then have the inhaler indicated after each administration the number of IU's remaining so that the patient can manage their medication usage and guarantee that they are receiving the proper amount of medication dependent upon their needs.

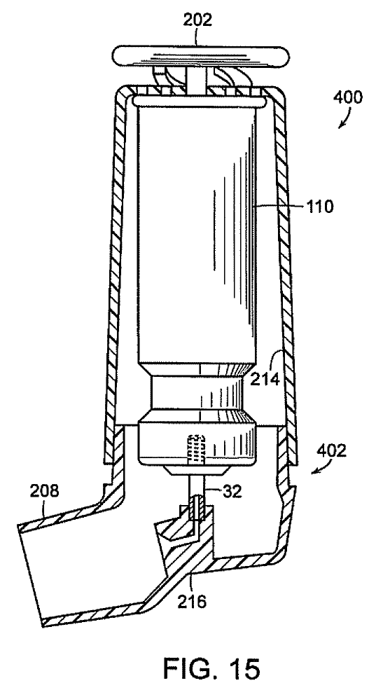

Another aspect of the instant application is shown in FIG. 15 where a traditional compression type inhaler 400 is shown. The inhaler 400 is comprised of a cap 214 and a base 216. Housed within the inhaler 400 is a canister 110 including a dose variable valve 10. At one end of the inhaler 400 is a mouthpiece 208. At the other end of the inhaler is a dial 202.

As shown in FIG. 15, the stem 32 of the dose variable valve 10 is secured in the base 216 by means of, for example, a key way. The canister 110 rests in the base 216, and a compression range 402 is maintained by the bias of the spring 72 which is internal to the canister and not shown in this view, but was discussed above with respect to FIG. 1. The bias of the spring 72 closes the dose variable valve 10, and also causes the canister 110 to act on the cap 214 to separate the cap 214 from the base 216 creating the compression range 402.

The dial 202 and the cap 214 includes markings. The cap 214 has a reference marking and the dial 202 includes dose selection markings. By aligning the desired dose selection marking with the reference marking the user can dial in a dose to be administered by the inhaler 400.

The dial 202 is mechanically connected to canister 110. As a result, a rotation of the dial 202 results in a rotation of the canister 110 in relation to the stem 32 which is essentially held in position by the key securing means. Thus rotation of the canister 110 about the stem 32 results in a change of the relationship of the metering chamber housing 18 and the variable metering plug 44 to change the volume of the metering chamber 100, which are shown in detail in FIGS. 1-5.

Once a dose is selected, the user can then depress the cap 214 in the direction of the base 216 to effectuate the release of the dose of medicament or drug stored in the metering chamber 100, through the stem 32 and out the mouthpiece.

One aspect of the inhaler 400 shown in FIG. 15 is that while it is preferred that the user be able to select the does as desired for self medication, the device is not so limited. The device may include for example, a locking mechanism, which would enable a doctor or pharmacist to set the dosing for the inhaler. In such a scenario, the doctor will write a prescription, for example for treatment of asthma. The treatment will require, for example, dosing of 50 μl as necessary. When the patient has the prescription filled the pharmacist can take a canister 110 having a dose variable valve 10 as described above and set the dosing in accordance with the prescription to deliver a 50 μl dose each time it is actuated.

According to one aspect of the invention, the lock could be part of the dial 202, and be re-settable. A key or unlocking mechanism may be provided to pharmacists to enable them to reset the inhaler 400 if for example, a subsequent prescription requires a larger dose be administered. In this manner, the patient does not have to purchase a new inhaler when receiving a new prescription. Alternatively, the lock could be part of the dose variable valve 10 and be a one time settable lock. Again this insures that the patient receives only the prescribed dose. Further, like the aspect above, the inhaler need not be replaced when transitioning to a new prescription, only the canister 110, which the pharmacists can set for the new prescription.

One benefit of such a device is that manufacturers need not prepare multiple different dosage canisters to be incorporated into an inhaler, rather, using a single inhaler a wide range of dosing parameters can be met.

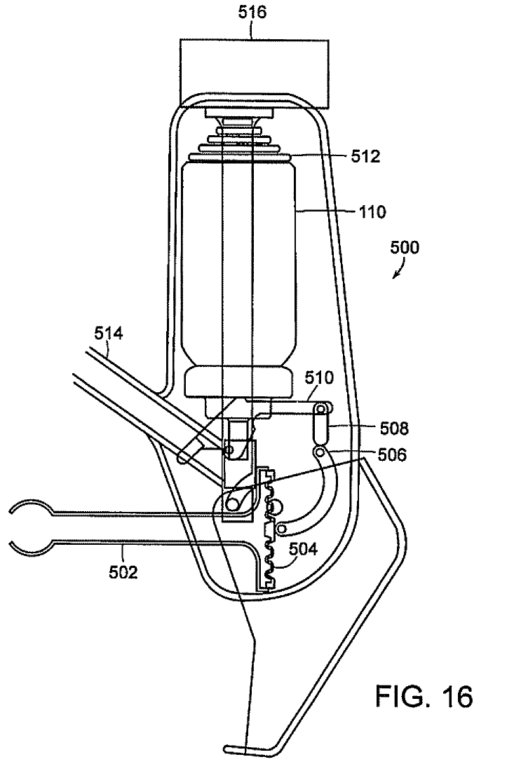

Yet a further aspect of the present invention can be seen in FIG. 16, where a breath actuated nasal drug delivery device 500 can be seen. Details of breath actuated nasal drug delivery devices can be found in commonly assigned and co-pending U.S. patent application Ser. No. 10/908,133, the contents of which are incorporated herein by reference. The nasal drug delivery device 500, operates in a similar fashion to that of the inhaler 200 described above, however in reverse. Rather than creating a vacuum in the inhaler, the user blows into the mouthpiece 502 to expand a diaphragm 504 causing a cam 506 to move, forcing a follower 508 to fall off of the cam 504 and permit a rocker 510 to rotate. The rotation of the rocker 510 allows a spring 512 to expand and release a dose of drug from the canister 110 into the nosepiece 514. As with the device described above with respect to FIGS. 6-14, a dial 516, in combination with a dose variable valve 10, housed within the canister 110 allows the user to set the proper dosing for the device.

Nasal delivery as described herein has been found effective for a variety of drug treatments and through the use of the Valsalva Maneuver, whereby the pulmonary tract of the user is essentially shut off from the nasal passages by the exhalation of the user. This prevents the particles being delivered to the user from simply avoiding the nasal region and ending up in the lungs. This can be an important aspect of treatment as it is well known that the nasal epithelia are effective organs for absorption of drugs into the body. Because of absorption via the nasal epithelia bypasses the blood brain barrier, it is one of the most effective methods of transmitting systemic pharmaceutical products, for example insulin. Accordingly, the aspect of the instant invention shown in FIG. 16 provides for both a superior absorption of the drug by the patient, but also provides for the self titration of that drug by the user.

One significant benefit of the devices described by the instant invention is the ability to alter dose size. This is important not just for administration of the proper amount of size but also to administer this dose in a single administration. For example, in the case of chronic obstructive pulmonary disease (COPD) and asthma, traditional dosing mechanisms are not always effective and may require the use of two or more inhalers providing different sized doses, or alternatively multiple doses from a single inhaler. This multiple dosing can lead to exhaustion on the part of the patient, who already has a diminished lung capacity and strength.

What is more, these multiple dosings may require multiple steps including shaking the inhaler between each administration as well as device preparatory steps or waiting between administrations. As a result, patients sometimes forget how many doses they have administered and often administer incomplete doses. This is quite understandable as these patients are often in the midst of an attack where breathing has become very difficult and their only goal is immediate relief.

The inhaler 200, for example, as shown in FIG. 6 allows patients to configure their dose as needed. Instead of taking multiple doses of a set strength, patients “dial-in” the appropriate dose they need, which is then administered in a single puff. This renders the need for separate inhalers redundant and alleviates the need for exhausting and often ineffective multiple inhalations.

Though some aspects of the instant invention are directed to providing consistent and repeatable doses to the pulmonary and/or nasal mucosa for delivery of insulin, the invention is not so limited and can be used for the treatment of any illness requiring either systemic or topical dosing. In addition to insulin and other medicaments for the treatment of diabetes, a variety of different types of formulations with bronchodilators can also be used in connection with the inhalers of the present invention.

Bronchodilators are intended to improve bronchial airflow and treatment of bronchial asthma is the most common application of these drugs. In addition to asthma bronchodilators help expand the airways and improve the breathing capacity of patients with emphysema, pneumonia and bronchitis. Salbutamol is a type of bronchodilator that can be used to aid in the treatment of chronic COPD and asthma that is characterized by the obstruction of airflow out of the lungs. Salbutamol being a short-acting β2-adrenergic receptor agonist binds to β2-adrenergic receptors leading to their activation thus relaxing the bronchial smooth muscles and widening the airways. Formoterol, a newer β2-adrenergic receptor agonist, aids in the same bronchodilation as salbutamol, but is long-acting. Duration of the effect of formoterol lasts about 12 hours, in comparison with 4-6 hours with salbutamol and it also has a faster onset than the latter.

Corticosteroids, which are the steroid hormones produced by the adrenal cortex, can also be put into formulations to be used in conjunction with the invention. Budesonide and fluticasone, which are types corticosteroids that have anti-inflammatory properties and are widely used against many inflammatory conditions, particularly for the treatment of asthma, non-infectious rhinitis. Formulations with a combination consisting of a corticosteroid (budesonide) and a bronchodilator (formoterol), which have been shown to have high efficacy, can also be used.

It is preferred that active ingredients in the formulations used in an inhaler as shown in FIG. 6 are readily made as suspensions within the propellant will be HFA-134(a), which is highly volatile. The high volatility of the propellant makes it possible to produce aerosolized forms of the active ingredients, which can then be inhaled by the patients. Various types of excipients or stabilizing agents, which are themselves inert and merely act as vehicles for the active ingredient, may also have to be used in the formulations to prevent flocculation. Some of the most common types of excipients that are used in different aerosol formulations include oleic acid, aspartame, water, ethanol, ethanolic acid, phosphatidyl choline etc. The excipients of choice will be the ones that give the most stable formulations.

While certain formulations and diseases have been specifically discussed herein, the present invention is not so limited and may be used with any formulation deliverable with a metered dose inhaler.

Thus by the foregoing examples, the objects and advantages of the present invention are realized, and although preferred embodiments have been disclosed and described in detail herein, its scope and objects should not be limited thereby; rather its scope should be determined by that of the appended claims.

What is claimed is:

1. A variable volume medicament valve comprising:
a. a housing; and
b. a plug insertable into said housing wherein a volume of medicament to be released by said valve is defined by a distance between a lower surface of the plug and a top surface of the housing and wherein said volume is variable.

2. The valve of claim 1 further comprising a stem having at least one orifice and a lumen therethrough, wherein said stem is translatable between a first and a second position and rotatable.

3. The valve of claim 2, wherein the first position is an at-rest position and a second position is a medicament dispensing position.

4. The valve of claim 2, wherein movement of said stem from said first to said second position places the orifice and lumen in fluid communication with the volume of medicament to be dispensed.

5. The valve of claim 2, wherein the stem interconnects with the plug such that rotation of the stem causes rotation of the plug and thereby variation of the volume of medicament to be dispensed.

6. A variable dose metered inhaler comprising:
a. an inhaler body;
b. a canister for storage of one or more pressurized doses of a medicament which is placed in the inhaler body; and
c. a dose variable valve, said dose variable valve including a valve housing and a valve plug, wherein a volume of drug to be administered can be varied by movement of the valve plug in relation to the valve housing.

7. The variable dose metered inhaler of claim 6 further comprising a valve stem interconnecting the canister, a metering chamber and the atmosphere.

8. The variable dose metered inhaler of claim 7, wherein the metering chamber is defined by a lower surface of the valve plug and an upper surface of the valve housing.

9. The variable dose metered inhaler of claim 6, wherein the valve plug and the valve housing are threaded together.

10. The variable dose metered inhaler of claim 6, further comprising a rotatable valve stem interconnected with said valve plug, wherein rotation of the valve stem causes the valve plug to move in relation to the valve housing which alters the volume of medicament to be administered.

11. A breath actuated variable dose metered inhaler comprising:
a. an inhaler body having a mouth piece;
b. a canister for storage of one or more pressurized doses of a medicament which is placed in the inhaler body;
c. a dose variable valve, said dose variable valve including a valve housing and a valve plug, wherein a volume of drug to be administered can be varied by movement of the valve plug in relation to the valve housing; and
d. a breath actuated trigger which releases the volume of drug to be administered to the mouthpiece upon inhalation by a user.

12. The breath actuated variable dose metered inhaler of claim 1 further comprising a valve stem interconnecting the canister, a metering chamber and the atmosphere.

13. The breath actuated variable dose metered inhaler of claim 12, wherein the metering chamber is defined by a lower surface of the valve plug and an upper surface of the valve housing.

14. The breath actuated variable dose metered inhaler of claim 11, wherein the valve plug and the valve housing are threaded together.

15. The breath actuated variable dose metered inhaler of claim 11, further comprising a rotatable valve stern interconnected with said valve plug, wherein rotation of the valve stem causes the valve plug to move in relation to the valve housing and to alter the volume of medicament to be administered.

16. The breath actuated variable dose metered inhaler of claim 11, further comprising an LED indicating when the desired volume of drug has been administered.

17. The breath actuated variable dose metered inhaler of claim 11, further comprising an inhalation units remaining indicator, wherein upon administration of a desired volume of drug the inhalation units remaining is updated to provide accurate accounting of the amount of drug in the canister.

18. The breath actuated variable dose metered inhaler of claim 11, further comprising a dial indicating to the user an amount of drug to be administered.

19. The breath actuated variable dose metered inhaler of claim 18, further comprising a computer, said computer extinguishing the LED upon the running of a clock function maintained by the computer and initialized by an actuation sensor.

20. The breath actuated variable dose metered inhaler of claim 19, wherein the dial provides a signal to the computer indicating the number of inhalation units the inhaler has been set for.

21. The breath actuated variable dose metered inhaler of claim 19, further comprising a cocking switch providing a signal upon the cocking of the inhaler.

22. The breath actuated variable dose metered inhaler of claim 11, further comprising a spring actuation mechanism.

23. The breath actuated variable dose metered inhaler of claim 11 further comprising a cocking mechanism altering the inhaler from an at-rest state to a ready to a cocked state.

24. The breath actuated variable dose metered inhaler of claim 11, wherein the breath actuated trigger includes a diaphragm which is deformed upon inhalation and triggers the release of the volume of drug to be administered.

25. A variable dose metered nasal drug delivery device comprising:
a. a nasal drug delivery device body having a mouthpiece and a nose piece;
b. a canister for storage of one or more pressurized doses of a medicament;
c. a dose variable valve, said dose variable valve including a valve housing and a valve plug, wherein a volume of drug to be administered can be varied by movement of the valve plug ill relation to the valve housing; and
d. a breath actuated trigger which releases the volume of drug to be administered to the nosepiece upon the exhalation of a user into the mouthpiece.

26. The variable dose metered nasal drug delivery device of claim 25 further comprising a valve stem interconnecting the canister, a metering chamber and the atmosphere.

27. The variable dose metered nasal drug delivery device of claim 26, wherein the metering chamber is defined by a lower surface of the valve plug and an upper surface of the valve housing.

28. The variable dose metered nasal drug delivery device of claim 25, wherein the valve plug and the valve housing are threaded together.

29. The variable dose metered nasal drug delivery device of claim 25, further comprising a rotatable valve stem interconnected with said valve plug, wherein rotation of the valve stem causes the valve plug to move in relation to the valve housing and to alter the volume of medicament to be administered.

30. A method of treatment of a condition in need thereof by application of a drug effective for the treatment thereof comprising steps of:
a. providing an inhaler;
b. providing a pressurized container having a medication;
c. setting a dose based upon the need of the patient;
d. inhaling on a mouthpiece of the inhaler; and
e. delivering the set dose to the patient.

31. The method of claim 30 further comprising a step of deducting the set dose from an inhalation unit's remaining counter.

32. The method of claim 30, further comprising:
a. providing a variable volume medicament valve having a housing, and a plug insertable into the housing wherein the set dose is defined by a distance between a lower surface of the plug and a top surface of the housing.

33. The method according to claim 32 further comprising a step of:
rotating the housing relative to the plug to change the distance and alter the size of the dose.

34. The method according to claim 32, wherein the patient sets the dose at the time of delivery.

35. The method according to claim 34, wherein the dose is variable dependent upon daily titration requirements.

36. The method according to claim 32, wherein a doctor sets the dose.

37. The method according to claim 32, wherein upon completion of the setting step the inhaler becomes a set-dose inhaler.

38. The method according to claim 32, wherein the metered dose inhaler is breath actuated.

39. A method of varying the volume of a valve for release of a pressurized material comprising: providing a variable volume medicament valve having a housing, and a plug insertable into the housing wherein a volume of medicament to be released by said valve is defined by a distance between a lower surface of the plug and a top surface of the housing; and rotating the housing relative to the plug to change the distance.
